(19) United States
(12) Patent Application Publication
(10) Pub. No.: US 2008/0243030 A1
(43) Pub. Date: Oct. 2, 2008

(54) MULTIFUNCTION CANNULA TOOLS

(75) Inventors: Eric J. Seibel, Seattle, WA (US); Charles David Melville, Issaquah, WA (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 11/695,175

(22) Filed: Apr. 2, 2007

(51) Int. Cl. A61B 10/02

(52) U.S. Cl. 600/566

(57) ABSTRACT

Different exemplary embodiments of cannula tools each perform multiple functions. Each cannula tool is able to dislodge or cut away a biopsy sample from a desired site within a patient's body, and then collect the sample. The cannula tool, which is disposed at the distal end of an elongate flexible tube is guided to the desired site over a guide wire. The cannula tool either abrades cells from adjacent tissue with an abrasive surface, or cuts away a sample of tissue with a sharpened cutting edge or with a loop that is electrically heated or pulled to snare the sample. The biopsy sample is then drawn with a bodily fluid or introduced fluid through an annulus formed between the guide wire and the inner surface of the elongate flexible tube or through a lumen, for collection at the proximal end of the elongate flexible tube.

### BACKGROUND

Catheters with cannula tools that are introduced into a patient's body by means of slipping the catheter with its cannula tools over a guidewire that has been maneuvered to a desired location in the body are well developed for cardiovascular applications. In these applications, the task of taking a biopsy for disease diagnosis was not required, so tools for cell sampling have not previously been developed. In contrast, one of the primary purposes of endoscopy and bronchoscopy is disease diagnosis, which often requires taking cell samples and tissue biopsies. It is likely that catheter-based or guidewire-based medical devices will be useful in the endoscopy and bronchoscopy fields, along with urology and other medical fields that require cell sampling for disease diagnosis. Catheters are inherently more flexible and smaller in diameter than endoscopes, so regions previously unexplored by endoscopes can be accessed by guidewire-based tools. It would clearly be desirable to develop a variety of different types of catheter-based or guidewire-based tools for use in collecting samples from an internal site in a patient's body for cytopathological diagnosis.

It will also be desirable to develop multifunctional cannula tools that can be employed to carry out more than one function, for example, dislodging cells and tissue, and then capturing and withdrawing the cells and tissue for diagnostic evaluation, to detect disease by applying conventional cytological and pathological procedures.

As is typically done, it is expected that a guidewire would be inserted into the body of a patient in a generally conventional manner advanced to a desired location where a biopsy of cells and tissue is to be taken. The process of advancing the guidewire can be done purely by exercising the touch and feel of an experienced physician, or can be carried out with visualization technologies, such as fluoroscopy, X-ray or computed tomography (CT) imaging, magnetic resonance imaging (MRI), ultrasound imaging, optical tomography, etc. After the guidewire has been inserted and advanced to a desired site, it would be desirable to introduce a multifunction tool over the guidewire as a cannula, or otherwise couple the tool to the guidewire so that it can be advanced to the site over the guidewire and be employed to obtain a biopsy sample at the site.

### SUMMARY

One aspect of the novel approach discussed below is directed to various exemplary embodiments of a cannula tool system for taking a biopsy sample of cells or tissue from an internal site with a body of a patient. In each of these exemplary embodiments, the cannula tool system includes an elongate tubular member having at least one lumen that is sized and configured to slip longitudinally over a guide that has been inserted into a body of a patient. The elongate tubular member can then be controllably advanced over the guide to a desired internal site within the body of the patient. Means associated with the elongate tubular member are provided for dislodging a biopsy sample from a desired internal site within a body of a patient. Also associated with the elongate tubular member are means for withdrawing the biopsy sample from a body of a patient for cytopathological evaluation.

The elongate tubular member generally extends between a proximal end and a distal end. The means for withdrawing can include a pump that is coupled in fluid communication with a lumen formed within the elongate tubular member. This pump is configured for extracting the biopsy sample through the lumen, from the distal end of the elongate tubular member, for example, by drawing the biopsy sample along with a bodily fluid through the lumen. The biopsy sample and bodily fluid are thus drawn toward the proximal end of the elongate tubular member where the biopsy sample can be collected for further study or evaluation.

The elongate tubular member has an exterior surface, and in one exemplary embodiment, the means for dislodging comprises a plurality of outwardly extending abrasive points disposed at spaced-apart positions on the exterior surface of the elongate tubular member and proximate to its distal end. These abrasive points are provided for abrading cells from the internal site within a body of a patient, and the cells that are abraded free of the tissue comprise the biopsy sample.

In addition, the means for withdrawing can further include a plurality of orifices formed in the elongate tubular member, proximate to the spaced-apart positions where the plurality of outwardly extending abrasive point are disposed. These orifices provide fluid communication paths for cells conveyed with a bodily fluid, so that the cells pass into the lumen of the elongate tubular member and are drawn by the pump through the lumen toward the proximal end of the elongate tubular member.

An annular gap is formed between the guide and an interior surface of the lumen and comprises a fluid path, so that cells passing through the plurality of orifices are conveyed by the bodily fluid through the annular gap to the proximal end of the elongate tubular member.

In another exemplary embodiment, the means for dislodging comprises a snare loop that extends generally distally of the distal end of the elongate tubular member. The snare loop can be employed to cut free a biopsy sample from an internal site within a body of a patient, by snaring the tissue. In one embodiment, the snare loop is coupled to a power supply that is selectively activated to heat the snare loop with an electrical current to a temperature sufficiently high to cut through tissue, freeing the biopsy sample from adjacent tissue. The biopsy sample that is freed from the adjacent tissue is then drawn with bodily fluid into and through the lumen of the elongate tubular member. In another embodiment, the snare loop is coupled to one or more lines or wires that extend proximally of the elongate tubular member. These one or more lines are pulled to tighten the snare loop around tissue, so that the loop cuts the tissue free from adjacent tissue. The tissue that is freed from the adjacent tissue can then be drawn into and through the elongate tubular member toward the proximal end of the elongate tubular member, to serve as a biopsy sample.

In still another exemplary embodiment, the means for dislodging comprises a helical member having a cutting blade formed on its distal end. The helical member is disposed in the lumen of the elongate tubular member, within an annular gap defined between an interior surface of the elongate tubular member and the guide. Rotation of the elongate tubular member about the guide causes the cutting blade to cut a piece of tissue free from adjacent tissue at an internal site within the body of a patient. The piece of tissue comprising the biopsy sample can then be drawn into and through the annular gap toward the proximal end of the elongate tubular member.

The cannula tool system can further include a prime mover and a rotational driver that is configured to drivingly couple with the elongate tubular member where it is exposed outside a body of a patient. The prime mover causes the rotational driver to rotate the elongate tubular member about the guide so that the cutting blade is rotated into tissue, cutting away a piece of tissue for use as a biopsy sample.

A biopsy trap can be disposed between the pump and the proximal end of the elongate tubular member, and in fluid communication with both. The biopsy trap serves to trap a biopsy sample that has passed through the lumen of the elongate tubular member.

In yet another exemplary embodiment, the means for dislodging comprises a balloon that is disposed around an exterior of the elongate tubular member and adjacent to its distal end. An outer surface of the balloon has an abrasive coating so that when the balloon is selectively inflated while the distal end of the elongate tubular member is disposed at an internal site with a body of a patient, and the elongate tubular member is then moved while the abrasive coating is in contact with tissue at the internal site, cells are dislodged from the tissue by the abrasive coating. These cells can then be drawn with bodily fluid into and through the elongate tubular member, e.g., through an annular gap defined between an interior surface of the elongate tubular member and the guide, and toward the proximal end by the pump.

In some exemplary embodiments, the elongate tubular member can include a guide collar disposed proximate to the distal end of the elongate tubular member, attached to one side of the elongate tubular member. The guide collar has a second lumen formed within it, and the second lumen is sized and configured to slide over the guide. The guide can comprise an elongate flexible member such as an endoscope, a catheter, or a flexible guidewire.

In at least one exemplary embodiment, the distal end of the elongate tubular member includes a sharp cutting edge that slices a piece of tissue comprising the biopsy sample free from adjacent tissue. This biopsy sample is conveyed by the pump into and through the lumen, toward the proximal end of the elongate tubular member.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A is a schematic block diagram of an exemplary multifunctional cannula tool system for collecting a biopsy sample from an internal site in a body of a patient;

FIG. 1B is an enlarged cut-away schematic view of an exemplary embodiment of a distal end of a cannula tool that includes an abrasive surface for abrading cells from adjacent tissue;

FIG. 2 is an enlarged cut-away schematic view of a distal end of an exemplary embodiment of a cannula tool that includes a loop for snaring tissue that is cut away for a biopsy sample;

FIG. 3 is an enlarged cut-away schematic view of a distal end of an exemplary embodiment of a cannula tool that includes a helical ribbon having a cutting edge for cutting away a piece of tissue for a biopsy sample;

FIG. 4 is an exploded view of an exemplary rotational driver that is disposed externally and is configured to drivingly rotate a cannula tool about a longitudinal axis;

FIG. 5 is a schematic view of an exemplary embodiment of a cannula tool having an inflatable balloon with an abrasive surface, within a body lumen, illustrating the inflatable balloon in a deflated state;

FIG. 6 is a schematic view of the exemplary embodiment of FIG. 5 after the balloon has been selectively inflated and either rotated or moved longitudinally so that the abrasive surface frees cells from tissue on the inner surface of the body lumen, enabling the cells to be drawn into an annular passage between a guidewire and inner surface of the cannula lumen; and

FIG. 7 is a schematic view of an exemplary embodiment of a cannula tool that includes a piggyback guide collar that is adapted to slide over a guidewire, and which has a cutting edge on a leading distal end of a lumen, so that a piece of tissue cut from tissue can be drawn into the lumen for collection at the proximal end of the cannula tool.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

### Exemplary Cannula Tool System

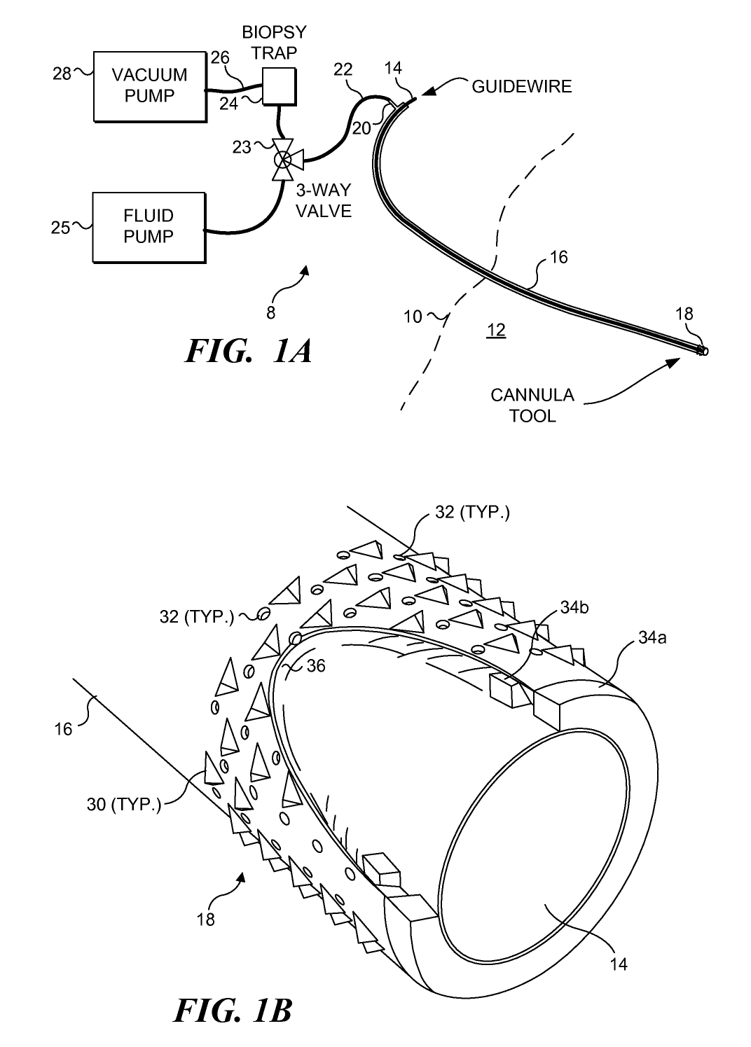

FIG. 1A illustrates an exemplary cannula tool system 8 that includes a first example of a cannula tool 18. In this schematic illustration, an elongate flexible tube 16 is illustrated extending through a dermal interface 10 and into a body 12 of a patient. Elongate flexible tube 16 is guided to a desired location within body 12 by a flexible guide wire 14, which has been inserted through an incision or other opening in dermal interface 10 and has already been advanced to the desired location or site. Elongate flexible tube 16 is then slid over flexible guide wire 14 and is thus guided to the desired location.

After thus being advanced to the desired location, cannula tool 18 can be employed to carry out a plurality of functions. Specifically, cannula tool 18 is employed to dislodge cells or tissue at the desired location, and then facilitates withdrawal of the dislodged cells or tissue from the site as a biopsy sample, for collection to enable further processing or analysis. The dislodged cells or tissue are conveyed from the site at the distal end of the elongate flexible tube through an annulus formed between guide wire 14 and the interior surface of elongate flexible tube 16, toward the proximal end of the elongate flexible tube. In this exemplary embodiment, a port 20 is formed at or adjacent to the proximal end of elongate flexible tube 16, in fluid communication with the annulus, and is coupled to one end of a fluid line 22. The other end of fluid line 22 is connected to a three-way valve 23, which selectively provides direct communication to a vacuum pump 28 for applying negative pressures or to a fluid pump 25 for applying positive pressures. Optionally, a biopsy trap 24 is disposed between the three-way valve and vacuum pump (or other source of a vacuum). The vacuum pump 28 coupled through a fluid line 26 to the opposite side of biopsy trap 24 produces a negative pressure that draws the biopsy sample, e.g., along with a bodily fluid such as blood, mucus, or an introduced fluid (e.g., air or saline), into the biopsy trap. The biopsy sample can then be removed from the biopsy trap so that the processing and analysis can be carried out. Fluid pump 25 can provide saline solution (from a source reservoir—not shown) to suspend the dislodged cells or tissue for easier retrieval. Furthermore, the introduced fluid under positive pressure can help to dislodge cells, mucus, blood, or other body fluid from the tool, guidewire, or wall of the body lumen.

Further details of cannula tool 18 are illustrated in FIG. 1B. Cannula tool 18, which is disposed at the distal end of elongate flexible tube 16, includes a plurality of spaced-apart outwardly extending points 30 disposed around the exterior surface of the elongate flexible tube. Alternatively, cannula tool 18 can instead be simply coupled to the distal end of elongate flexible tube 16 through an appropriate coupling mechanism (not shown). Points 30 are shaped so that they tend to abrade cells from tissue adjacent to the outer surface on which the points are disposed when the elongate flexible tube is moved longitudinally back-and-forth a few centimeters (and/or rotated back and forth around) its longitudinal axis. A plurality of orifices 32 are interspersed between the plurality of points providing a number of fluid communication paths through which the bodily fluid and cells dislodged from the adjacent tissue can be drawn into an annulus 36, which is disposed between the outer surface of guide wire 14 in the inner surface of elongate flexible tube 16. As explained above, the bodily fluid conveys the dislodged cells or tissue through annulus 36 from the distal end of the elongate flexible tube to the proximal end where port 20 is disposed, so that the cells or tissue comprising the biopsy sample can be drawn into biopsy trap 24 and collected.

A seal comprising annular rings 34a and 34b is disposed at the distal end of elongate flexible tube 16 to ensure that vacuum pump 28 draws the bodily fluid and dislodged cells or tissue through orifices 32, rather than simply drawing bodily fluid without the cells or tissue from around guide wire 14 at the distal end of the elongate flexible tube. It will be understood that the shape or configuration of points 30 are intended to be exemplary and not in any way limiting, since it should be apparent that a number of other different shapes or configurations can be employed for such points comprising an abrasive surface, such as short bristles (e.g., a tubular brush). Further, either more or fewer points 30 can be disposed on cannula tool 18, over either a longer or shorter length section.

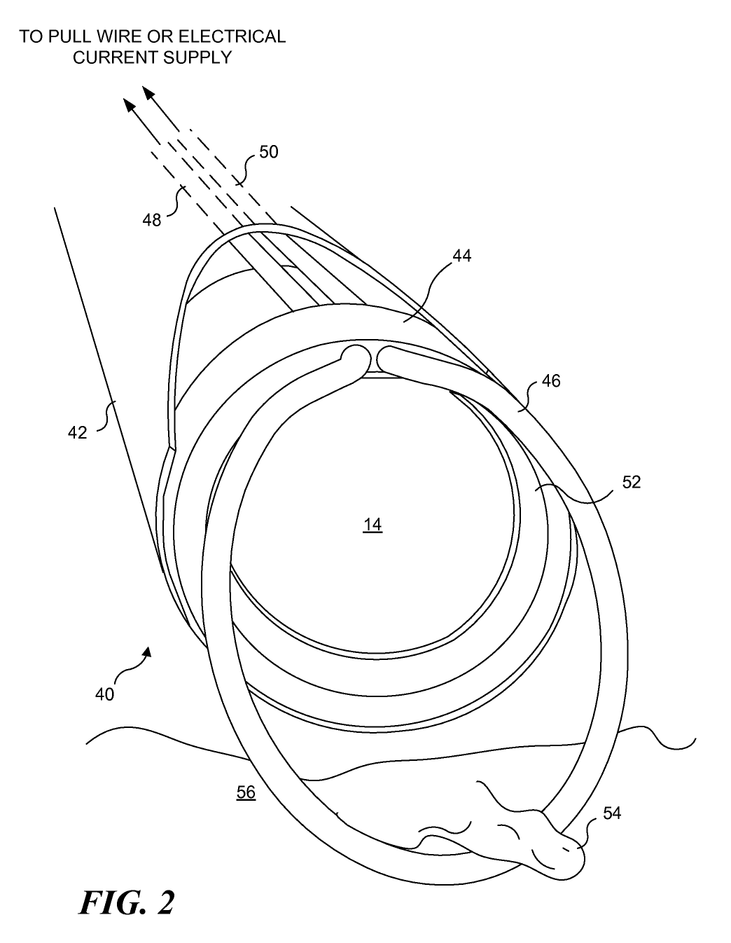

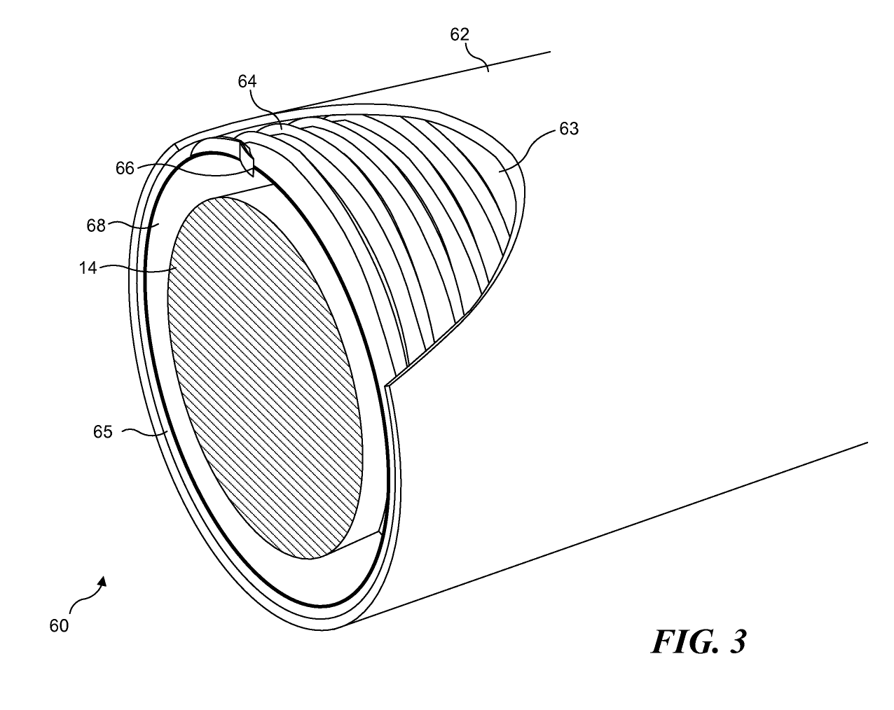

In FIG. 2, an exemplary cannula tool 40 is illustrated for use in connection with an elongate flexible tube 42. This same outer tube can also be used to shield the internal tissues of the body from cannula tool 18 during insertion to the desired location within the body. This outer elongate flexible tube or sheathing can then be withdrawn to reveal the biopsy tool distal tip when it is disposed at the desired biopsy site within the body. As in cannula tool 18, cannula tool 40 is configured to be advanced to a desired site by sliding along guide wire 14. Cannula tool 40 includes a wire loop 46 that extends distally of a ring 44, which is disposed just inside the distal end of elongate flexible tube 42. Loop 46 is sized to ensnare a biopsy sample of tissue, such as a polyp 54 growing from adjoining tissue 56 at the desired site, as shown in this Figure.

Two different techniques can be employed to cut away the piece of tissue comprising the biopsy sample from adjoining tissue. One option is to supply an electrical current so that loop 46 is heated sufficiently to burn through adjacent tissue 56, freeing the biopsy sample, such as polyp 54. The electrical current can be applied to loop 46 through conductive wires 48 and 50, which extend proximally of the elongate flexible tube 42 and are connected through a switch to a conventional electrical current supply (neither shown). Alternatively, one or both of wires 48 and 50 can be pulled proximally of the proximal end of elongate flexible tube 42, which tightens loop 46 around the tissue sufficiently to cut through the tissue, freeing it from the adjacent tissue. To help stabilize the tool over the sample and possibly help to ensnare the tissue, a vacuum can be applied within outer elongate flexible tube 42. Once the biopsy sample is freed, it can be drawn with bodily fluid into an annulus 52 formed between the outer surface of guide wire 14 and the inner surface of the elongate flexible tube 42. For example, vacuum pump 28 (shown in FIG. 1A) can be used to draw the bodily fluid and the dislodged tissue comprising the biopsy sample to the proximal end of the elongate flexible tube 42. This biopsy sample that has thus been freed and withdrawn from inside the body of a patient can then be collected for processing and analysis by medical personnel.

An exemplary embodiment of a cannula tool 60 shown in FIG. 3 is also configured to be advanced to desired site within the body of the patient over guide wire 14. Cannula tool 60 includes an outer elongate flexible tube 62 that extends between a distal end and a proximal end. The proximal end is disposed outside the body of the patient. Within the outer elongate flexible tube is disposed a middle flexible tube 65. On the outer surface of the middle flexible tube, at its distal end, is affixed a helical coil 64. The helical coil is in contact with the inner surface of the outer elongate flexible tube and terminates on its leading end at a sharp cutting edge 66, which extends just beyond the distal end of elongate flexible tube 62. Sharp cutting edge 66 is thus configured to slice a ribbon of tissue from the desired site as middle flexible tube 65 is rotated about its longitudinal axis in the appropriate direction with respect to outer elongate flexible tube 62 to bring the sharp cutting edge into the adjacent tissue. This ribbon of tissue, which comprises a biopsy sample, is carried between helical coils 64 and conveyed with bodily or introduced fluid through an annulus 68 formed between the outer surface of guide wire 14 and the inner surface of middle flexible tube 65. A smaller annular gap 63 is provided between the outer surface of middle flexible tube 65 and the inner surface of outer elongate flexible tube 62, which may facilitate capture of the tissue ribbon when negative pressure is applied at the proximal end of the elongate flexible tube or may facilitate removal of the tissue ribbon from the helical coil with introduced fluid applied at the proximal end. As discussed above, the vacuum pump shown in FIG. 1A can be used to draw the biopsy sample and bodily fluid through the annulus toward the proximal end of the elongate flexible tube, where the biopsy sample can be collected.

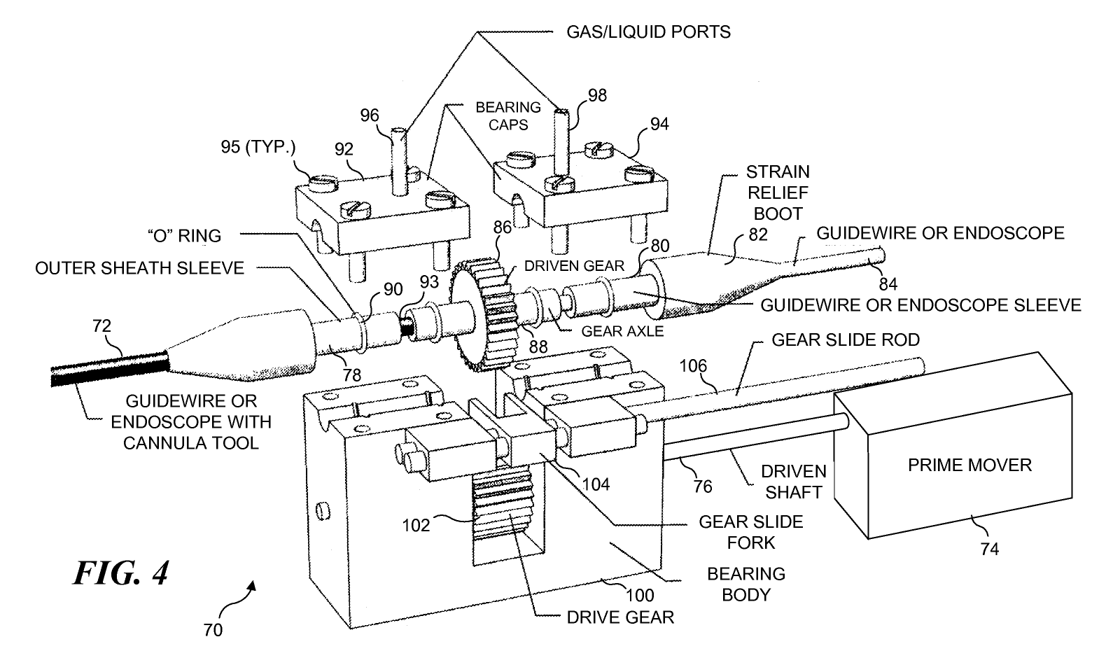

FIG. 4 illustrates a rotational driver 70 that is configured to rotate a guidewire or endoscope 72 about a longitudinal axis of the device. Attached to the distal end of the guidewire or endoscope can be one of the multifunctional cannula tools discussed herein. The endoscope should be viewed as a specific type of “elongate flexible tube,” which is used with each of the exemplary embodiments of the cannula tools discussed herein. However, this exemplary embodiment matches the ribbon cutting tool in FIG. 3, which optionally can provide fluid communication in both annular gaps around the middle flexible tube. Note that an endoscope can also serve as a guide wire with eyes.

In connection with rotational driver 70, a prime mover 74 (for example, an electric motor) is included to rotate a driven shaft 76, thereby providing a rotational force that rotates and drives middle flexible tube 65, which holds the biopsy tool, forward and backward. An outer sheath sleeve 78 is fitted over guidewire or endoscope 72. Sealing “O” rings 90 are provided on outer sheath sleeve 78, as well as on each side 88 of a driven gear 86, and on a guidewire or endoscope sleeve 80, which is near a proximal end 84 of the endoscope and attached to a strain relief boot 82. End caps 92 and 94 are fitted over and sealingly engage “O” rings 90, when securely coupled to a bearing body 100 by fasteners 95. End caps 92 and 94 include ports 96 and 98, to provide fluid paths in fluid communication with exposed portions of the endoscope. At each of these exposed portions, the elongate flexible tube is open for withdrawing or injecting either gases or liquids into one or two annular gaps formed in guidewire or endoscope with cannula tool 72. When combining the components of FIGS. 3 and 4, port 98 can be used to withdraw a biopsy sample that is conveyed with a bodily or introduced fluid from the distal end of guidewire or endoscope with cannula tool 72, through annulus 68 formed between guide wire 14 and the inner surface of middle flexible tube 65. Port 96 is in fluid communication with small annular gap 63 that is formed between the outer surface of middle flexible tube 65 and the inner surface of outer elongate flexible tube 62. For example, a vacuum can be applied to both ports 96 and 98 in FIG. 4, which will help pull the tissue up against the cutting edge of the cannula tool in FIG. 3. After cutting is completed by advancing helical coil 64 on middle flexible tube 65 toward the tissue by rotating the drive gear that is mechanically coupled with the middle flexible tube, the vacuum can be removed from only the outer annular gap, i.e., from port 96. By applying a saline solution to port 96, any tissue and cells within the helical coil can be flushed and then sucked into the larger inner annulus 68, with negative pressure applied to port 98, e.g., with vacuum pump 25 (FIG. 1A).

Driven shaft 76 is drivingly coupled to a drive gear 102, which is rotatably mounted in bearing block 100 and affixed to an end of driven shaft 76. A gear slide fork 104 is mounted on the side of bearing block 100 and is configured to engage driven gear 86, so as to move the driven gear into meshing relationship with drive gear 102 when gear slide rod 106 is appropriate pushed (or pulled) longitudinally. By thus moving gear slide rod 106, a user can selectively engage driven gear 86 with drive gear 102 to apply a rotational force that begins turning driven gear 86, which is in mechanical communication with middle flexible tube 93, so that the middle flexible tube turns in one direction versus the opposite direction, about its longitudinal axis. Rotational motion of the cannula tool shaft can be used either for abrading or cutting cells and tissue from adjacent tissue at a desired location in a body of a patient. For example, the rotational driver can be used to rotate a cannula tool having an abrasive surface, such as exemplary cannula tool 18, or can turn a cannula tool that has a sharp cutting edge, which is able to cut away a ribbon of tissue to form a biopsy sample, such as exemplary cannula tool 60.

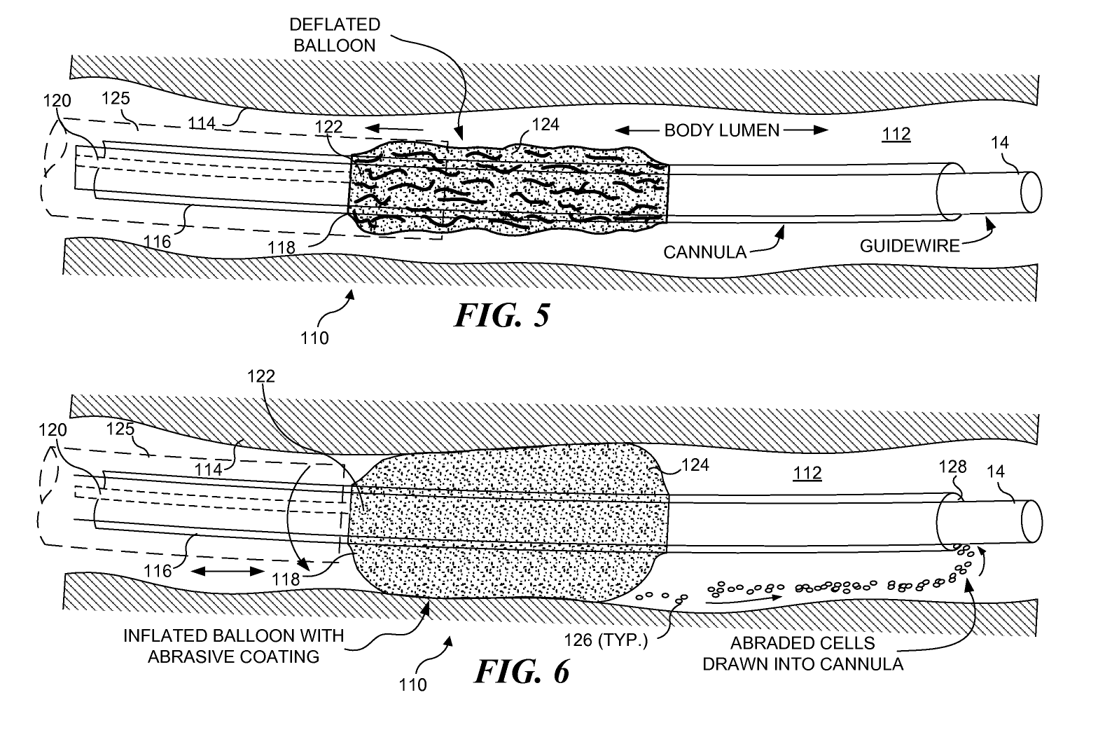

In FIG. 5 a cannula tool 110 is illustrated and is disposed adjacent to the distal end of a flexible elongate tube 116. As explained above, flexible elongate tube 116 has an internal lumen that is sized to be guided over guide wire 14 to a desired site within a body of a patient. For example, as shown in this Figure, guide wire 14 has been advanced through a body lumen 112 and has been used to guide elongate flexible tube 116 so that a balloon 118 (currently deflated) is advanced to a site where cells comprising a biopsy sample is to be taken. Balloon 118 is sealingly attached to the outer surface of the flexible elongate tube at each end of the balloon (for example, by thermal bonding or using an appropriate adhesive). A pressurized fluid tube 120 extends within an annulus formed between the outer surface of guide wire 14 and the inner surface of elongate flexible tube 116, from the proximal end of the flexible elongate tube to a port 122. Port 122 provides an opening through elongate flexible tube 116 to add or remove fluid volume inside balloon 118. The proximal end of the pressurized fluid tube is coupled to a pressurized fluid source, such as fluid pump 25, and to a negative pressure source, such as vacuum pump 28, both of which are shown in FIG. 1A.

Pressurized fluid provided by the pressurized fluid source can be selectively applied through pressurized fluid tube 120 and port 122 to inflate balloon 118, as shown in FIG. 6. When balloon 118 is thus inflated, an abrasive coating 124 that extends over the outer surface of balloon 118 comes into contact with tissue on the wall of body lumen 112. When the elongate flexible tube 116 is then rotated around its longitudinal axis or pushed/pulled longitudinally back-and-forth within body lumen 112, cells 126 are abraded from the tissue lining the body lumen by the abrasive coating. The abraded cells and bodily fluid within body lumen 112 are together drawn into an annulus 128 formed between the inner surface of the lumen extending through elongate flexible tube 116 and the outer surface of guide wire 14, as shown in FIG. 6. These cells, which comprise a biopsy sample, can be withdrawn from the annulus at the proximal end of flexible elongate tube 116, generally as explained above. An elongate sheath 125 (shown in FIGS. 5 and 6 after the elongate sheath has been pulled back) can be disposed around the abrasive balloon during insertion and retraction and then pulled back or proximally to expose the abrasive surface of the balloon when the cannula tool is disposed at a desired site for taking a biopsy sample. The proximal end of elongate sheath 125 is accessible outside the body of a patient and can be separately manipulated to move it longitudinally, relative to balloon 118 (or other embodiments of the cannula tool). For other types of cannula tool, it may be preferable to rotate elongate sheath about its longitudinal axis between first and second positions, so that an opening at its distal end (not shown) selectively either protects or exposes the portion of the cannula tool that is used to dislodge cells or tissue comprising the biopsy sample.

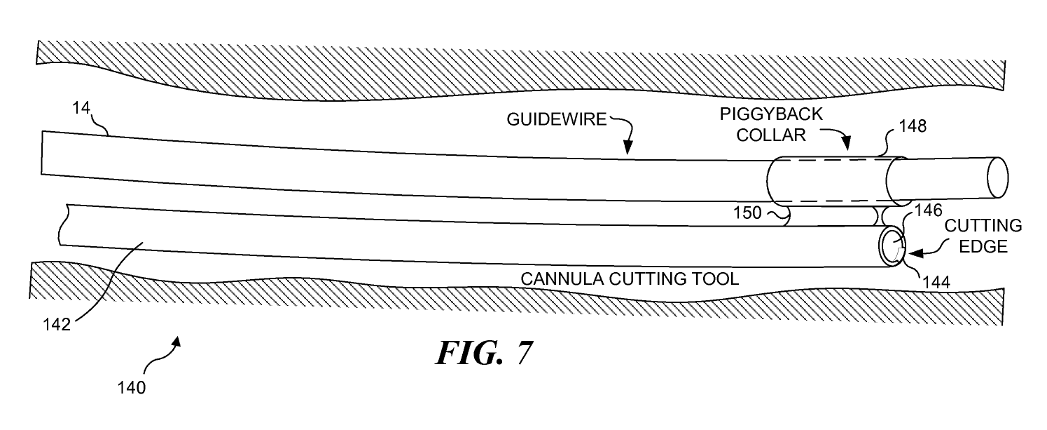

Yet another exemplary embodiment of a cannula tool 140 is illustrated in FIG. 7. Cannula tool 140 is intended to cut away a ribbon of tissue with a sharpened cutting edge 144, which is formed over at least a portion of the distal leading edge of the elongate flexible tube. Unlike the other exemplary embodiments of cannula tools discussed above, cannula tool 140 has a flexible elongate tube 142 with an internal lumen 146 that does not slide over guide wire 14. Lumen 146 instead defines a passage through which a ribbon of tissue that is cut away by sharpened cutting edge 144 from adjacent tissue within the body of a patient can be drawn with bodily fluid toward the proximal end of the elongate flexible tube. While not shown in this Figure, it is contemplated that a protective cover can be provided that overlies sharpened cutting edge 144 until the distal end of the cannula tool is disposed where it is desired to cut away a ribbon of tissue as a biopsy sample. A pull wire (not shown) that is coupled to the protective cover can then be pulled proximally, enabling the protective cover to be pulled away from the sharpened cutting edge and out through the proximal end of lumen 146. Alternatively, during insertion and retraction, flexible elongate tube 142 can be rotated so that cutting edge 144 is disposed close to the guidewire and away from the tissue, then rotated outwardly toward the tissue, to cut away the biopsy sample.

The distal end of the cannula cutting tool is guided to a desired location by a piggyback collar 148, which is attached to one side of the flexible elongate tube with a stanchion 150. Piggyback collar 148 includes an internal open guide lumen that is sized to readily slide along guide wire 14, and thus, to be guided to a desired site within the body of a patient where a biopsy sample is to be taken. Stanchion 150 may enable elongate tube 142 to rotate but prevent it from sliding longitudinally to enable cutting edge 144 to be turned away from the tissue during insertion and retraction of the tool through the body lumen.

Each of the exemplary embodiments of cannula tools discussed above is characterized by performing at least two functions. The first function is to dislodge or cutaway cells or tissue from within the body of a patient. The second function is to enable the cells or tissue that have been dislodged to be collected as a biopsy sample for further processing or analysis. A multi-functional capability and relatively compact size of these cannula tools enable them to be readily used in many applications where conventional cannula tools cannot be.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A cannula tool system for taking a sample of cells or tissue at an internal site within a body of a patient, comprising:
(a) an elongate tubular member having at least one lumen that is sized and configured to slip longitudinally over a guide that has been inserted into a body of a patient, so that the elongate tubular member is controllably advanced over the guide to a desired internal site within the body of the patient;
(b) means associated with the elongate tubular member for dislodging a biopsy sample from a desired internal site within a body of a patient; and
(c) means associated with the elongate tubular member for withdrawing the biopsy sample from a body of a patient for cytopathological evaluation.

2. The cannula tool system of claim 1, wherein the elongate tubular member extends between a proximal end and a distal end, wherein the means for withdrawing comprises a pump in fluid communication with a lumen formed within the elongate tubular member, said pump being configured for extracting the biopsy sample through the lumen from the distal end of the elongate tubular member by drawing the biopsy sample with a bodily fluid through the lumen, toward the proximal end of the elongate tubular member.

3. The cannula tool system of claim 2, wherein when the elongate tubular member is disposed proximate to tissue, the pump applies a negative pressure that draws the tissue toward or into the tube lumen.

4. The cannula tool system of claim 2, wherein the elongate tubular member has an exterior surface, and wherein the means for dislodging comprises a plurality of outwardly extending abrasive points disposed at spaced-apart positions on the exterior surface of the elongate tubular member, proximate to the distal end thereof, said abrasive points abrading cells from an internal site within a body of a patient.

5. The cannula tool system of claim 2, further comprising an annular gap that is defined between an interior surface of the lumen and the guide, wherein cells comprising the biopsy sample, which are dislodged from the internal site, are withdrawn from the internal site through the annular gap.

6. The cannula tool system of claim 5, wherein the annular gap is coupled in fluid communication with a source of a fluid that is introduced through the annular gap to dislodge and suspend the cells comprising the biopsy sample, through the annular gap.

7. The cannula tool system of claim 4, wherein the means for withdrawing further comprises a plurality of orifices formed in the elongate tubular member proximate to the spaced-apart positions where the plurality of outwardly extending abrasive points are disposed, so that the plurality of orifices provide fluid communication paths for cells conveyed with a bodily fluid to pass into the lumen of the elongate tubular member and be drawn by the pump through the lumen toward the proximal end of the elongate tubular member.

8. The cannula tool system of claim 7, wherein an annular gap is formed between the guide and an interior surface of the lumen, so that cells conveyed by bodily fluid are conveyed through the annular gap from the plurality of orifices to the proximal end of the elongate tubular member.

9. The cannula tool system of claim 2, wherein the means for dislodging comprises a snare loop that extends generally distal of the distal end of the elongate tubular member, the snare loop being employed to cut away tissue comprising the biopsy sample from an internal site within a body of a patient.

10. The cannula tool system of claim 9, wherein the pump applies a negative pressure to tissue that is adjacent to the snare loop, to draw said tissue into the snare loop while the snare loop has a larger diameter, so that the tissue drawn into the snare loop is cut away when the snare loop is drawn to a smaller diameter.

11. The cannula tool system of claim 9, wherein the snare loop is coupled to a power supply that is selectively activated to heat the snare loop with an electrical current sufficiently to cut through tissue, thereby freeing the biopsy sample from adjacent tissue, the tissue that is freed from the adjacent tissue being drawn with bodily fluid into and through the lumen of the elongate tubular member.

12. The cannula tool system of claim 9, wherein the snare loop is coupled to a line that extends proximally of the elongate tubular member and is pulled to tighten the snare loop around tissue to cut the tissue from adjacent tissue, so that the tissue freed from the adjacent tissue can be drawn into and through the elongate tubular member toward the proximal end of the elongate tubular member.

13. The cannula tool system of claim 2, wherein the means for dislodging comprises a helical member having a cutting blade formed on its distal end and disposed in the lumen of the elongate tubular member, within an annular gap defined between an interior surface of the elongate tubular member and the guide, rotation of the elongate tubular member about the guide causing the cutting blade to cut free a piece of tissue comprising the biopsy sample at an internal site within a body of a patient, the piece of tissue being then drawn into and through the annular gap toward the proximal end of the elongate tubular member.

14. The cannula tool system of claim 13, further comprising a prime mover, and a rotational driver that is configured to drivingly couple with the elongate tubular member where exposed outside a body of a patient, the prime mover causing the rotational driver to rotate the elongate tubular member about the guide so that the cutting blade is rotated into tissue to cut away the piece of tissue.

15. The cannula tool system of claim 13, further comprising an annular gap formed between an interior surface of the lumen and the guide, wherein the piece of tissue that is cut away is drawn through the annular gap by a negative pressure produced by the pump.

16. The cannula tool system of claim 2, further comprising a biopsy trap disposed between and in fluid communication with the pump and the proximal end of the elongate tubular member, the biopsy trap serving to trap the biopsy sample after it exits from the elongate tubular member.

17. The cannula tool system of claim 2, wherein the means for dislodging comprises a balloon that is disposed around an exterior of the elongate tubular member adjacent to the distal end of the elongate tubular member, an outer surface of the balloon having an abrasive coating so that when the balloon is selectively inflated while the distal end of the elongate tubular member is disposed at an internal site with a body of a patient, and the elongate tubular member is moved while the abrasive coating is in contact with tissue at the internal site, cells are dislodged from the tissue by the abrasive coating on the balloon and are drawn with the bodily fluid into and through the lumen of the elongate tubular member by the pump, toward the proximal end of the elongate tubular member.

18. The cannula tool system of claim 17, wherein the cells that are dislodged are drawn through an annular gap defined between an interior surface of the lumen and the guide.

19. The cannula tool system of claim 2, further comprising an outer elongate sheath that is movable between a first position that protects the means for dislodging during insertion and retraction of the elongate flexible member within a body of a patient, and a second position that exposes the means for dislodging the biopsy sample, when the means for dislodging is disposed at the desired internal site.

20. The cannula tool system of claim 19, wherein the outer elongate sheath extends proximally and is pulled proximally to achieve the second position in which the means for dislodging are exposed.

21. The cannula tool system of claim 19, wherein the outer elongate sheath extends proximally and is rotated relative to the cannula tool to achieve the second position in which the means for dislodging are exposed.

22. The cannula tool system of claim 2, wherein the elongate tubular member includes a guide collar disposed proximate to the distal end of the elongate tubular member, the guide collar being attached to one side of the elongate tubular member and having a second lumen formed within the guide collar, the second lumen comprising the at least one lumen that is sized and configured to slide over the guide.

23. The cannula tool system of claim 22, wherein the distal end of the elongate tubular member includes a sharp cutting edge that slices a piece of tissue free from adjacent tissue, the piece of tissue comprising the biopsy sample, the biopsy sample being conveyed by the pump, into and through the lumen, toward the proximal end of the elongate tubular member.

24. The cannula tool system of claim 1, wherein the guide comprises an elongate flexible member selected from a group consisting of:
(a) an endoscope;
(b) a catheter; and
(c) a flexible guidewire.

25. A multifunctional cannula tool for carrying out a plurality of functions, the multifunctional cannula tool comprising:
(a) an elongate tubular body having a proximal end that remains external to a body of a patient, and a distal end that is configured to be inserted into a body of a patient, the elongate tubular body including a guide lumen that is sized and configured to slide over a flexible guide member, to advance the distal end of the elongate tubular body to an internal site within a body of a patient;
(b) a dislodgment portion of the elongate tubular body that is configured to dislodge at least one of cells and tissue and which is disposed proximate to the distal end of the elongate tubular body; and
(c) a passage formed within the elongate tubular body and defining a fluid path through which said at least one of cells and tissue that are dislodged from the internal site to which the distal end of the elongate tubular body has been advanced, are drawn toward the proximal end of the elongate tubular body for collection as a biopsy sample.

26. The multifunctional cannula tool of claim 25, wherein the dislodgment portion comprises an abrasive surface that is configured to abrade cells from tissue that is adjacent to the distal end of the elongate tubular body, so that cells thus freed from the tissue can be drawn through the passage formed within the elongate tubular body and collected as a biopsy sample.

27. The multifunctional cannula tool of claim 26, further comprising a pressurized fluid lumen that is configured to convey a pressurized fluid to the distal end of the elongate tubular body from an external source of pressurized fluid, wherein the dislodgment portion further includes a selectively inflatable membrane on which the abrasive surface is disposed, the selectively inflatable membrane extending at least partially around a circumference of the elongate tubular body and being disposed adjacent to the distal end of the elongate tubular body, so that when the inflatable membrane is selectively expanded outwardly by being inflated with a pressurized fluid conveyed through the pressurized fluid lumen, and the elongate tubular body is then moved relative to tissue contacted by the abrasive surface, the abrasive surface on the inflatable membrane abrades cells from the tissue thus contacted by the abrasive surface, for collection as the biopsy sample.

28. The multifunctional cannula tool of claim 26, wherein the abrasive surface comprises a plurality of spaced-apart points that extend outwardly from an exterior of the elongate tubular body, near its distal end, and configured so that when the elongate tubular body is moved relative to tissue contacted by the spaced-apart points, said points abrade cells from the tissue contacted, freeing cells to be drawn into the passage and collected at the proximal end of the elongate tubular body as a biopsy sample.

29. The multifunctional cannula tool of claim 25, wherein the dislodgment portion comprises a loop disposed adjacent to the distal end of the elongate tubular body, said loop being configured and adapted to snare tissue disposed proximate thereto, detaching the tissue that is snared, so that the tissue can be drawn into and conveyed through the passage, and collected at the proximal end of the elongate tubular body as a biopsy sample.

30. The multifunctional cannula tool of claim 29, further comprising electrical leads that extend from the proximal end, distally along the elongate tubular body, and which are connected to the loop, proximal ends of said leads being adapted to connect to a power supply that selectively supplies an electrical current to the loop to heat the loop sufficiently to cut through tissue snared by the loop, so that the tissue is freed.

31. The multifunctional cannula tool of claim 29, wherein the loop is coupled to a wire that extends proximally of the elongate tubular body, an end of the wire that extends externally from the proximal end of the elongate tubular body being adapted to be pulled proximally to reduce a size of the loop so that the loop cuts free the tissue that is snared by the loop for collection as a biopsy sample.

32. The multifunctional cannula tool of claim 25, wherein the dislodgment portion comprises a cutting edge that is disposed adjacent to the distal end of the elongate tubular body, said cutting edge being adapted to cut tissue free for collection as a biopsy sample.

33. The multifunctional cannula tool of claim 32, wherein the cutting edge is disposed at a distal end of a helical corkscrew cutting blade that is disposed within the passage, so that tissue cut away with the cutting edge is drawn through the passage for collection at the proximal end of the elongate tubular body, as a biopsy sample.

34. The multifunctional cannula tool of claim 32, wherein the cutting edge is disposed at a leading distal edge of the passage, and wherein the guide lumen comprises a relatively shorter section of a tubular member that extends generally parallel to the elongate tubular body and is attached along a side of the passage.

35. The multifunctional cannula tool of claim 32, wherein the passage comprises an annular gap defined between an interior surface of the elongate tubular body and a flexible guidewire, a biopsy sample being withdrawn through the annular gap.

36. A method for using a cannula tool to carry out multiple functions within a body of a patient, comprising the steps of:
(a) inserting a flexible guide into the body of the patient and maneuvering a distal end of the flexible guide to a defined location;
(b) sliding a hollow cannula tool comprising an elongate tubular body over the flexible guide, until a distal end of the elongate tubular body is disposed at a desired site within the body of the patient;
(c) using a portion of the cannula tool disposed proximate to the distal end of the elongate tubular body, dislodging cells or tissue from the desired site; and
(d) at a proximal end of the elongate tubular body, collecting a biopsy sample comprising the cells or tissue that were dislodged, by drawing the cells or tissue that were dislodged through a passage formed in the cannula tool, toward a proximal end of the elongate tubular body, for withdrawal from the passage.

37. The method of claim 36, wherein the step of dislodging comprises the step of abrading cells from tissue within the body of the patient using an abrasive surface disposed adjacent to the distal end of the elongate tubular body.

38. The method of claim 37, wherein the abrasive surface comprises a plurality of sharp points that are spaced apart on an exterior of the elongate tubular body, adjacent to its distal end, further comprising the step of moving the elongate tubular body relative to adjacent tissue so the cells are abraded from and freed from the tissue by the plurality of sharp points.

39. The method of claim 37, wherein the abrasive surface is formed on a flexible membrane, further comprising the steps of:
(a) selectively expanding the flexible membrane outwardly until the abrasive surface on the flexible membrane contacts adjacent tissue; and
(b) then moving the abrasive surface on the elongate tubular body relative to the adjacent tissue, to abrade cells from the adjacent tissue.

40. The method of claim 36, wherein the step of dislodging comprises the step of snaring and cutting tissue from the desired site using a loop disposed at the distal end of the elongate tubular body.

41. The method of claim 40, wherein the step of snaring and cutting the tissue comprises the step of heating the loop to cut through the tissue that is snared, to free the tissue from adjacent tissue.

42. The method of claim 40, wherein the step of snaring and cutting the tissue comprises the step of reducing a size of the loop, causing the loop to cut away the tissue that is snared, to free said tissue from adjacent tissue.

43. The method of claim 36, further comprising the step of applying a negative pressure to a proximal portion of the cannula tool to withdraw tissue that has been dislodged at the distal end of the elongate tubular body, out through the passage.

44. The method of claim 36, further comprising the steps of selectively protecting the portion of the cannula tool when it is being advanced to the desired site; and selectively exposing the portion of the cannula tool to dislodge the biopsy sample at the desired site.

45. The method of claim 36, wherein the step of dislodging comprises the step of cutting away a piece of tissue from the desired site with a cutting blade disposed at the distal end of the elongate tubular body.
